(19) United States
(12) Patent Application Publication
(10) Pub. No.: US 2023/0137210 A1
(43) Pub. Date: May 4, 2023

(54) Cannabinoid Product for Assistance with Weight Loss

(71) Applicant: ECS Brands Ltd., Broomfield, CO (US)

(72) Inventor: Arthur Jaffee, Denver, CO (US)

(73) Assignee: ECS Brands Ltd, Broomfield, CO (US)

(21) Appl. No.: 17/514,387

(22) Filed: Oct. 29, 2021

(51) Int. Cl. A61K 31/352; A61K 31/05; A61K 47/44; A61P 3/04

(52) U.S. Cl. CPC A61K 31/352; A61K 31/05; A61K 47/44; A61P 3/04

(57) ABSTRACT

Cannabinoids are increasingly found to have beneficial effects on the human body, even without the intoxicating effects from THC. Herein we describe a cannabinoid which is used to assist with weight loss in the individual taking the cannabinoid. Tetrahydrocannabivarin, typically but not necessarily combined with cannabidiol and other assorted cannabinoids, provides a product for weight loss without the need for diet, exercise, pharmaceuticals, or surgery.

### BACKGROUND

### Field of the Invention

The present invention relates generally to cannabinoids which assist with weight loss. More specifically, the present invention relates to varin-rich cannabinoid extracts, such as tetrahydrocannabivarin, and its associated consumption for assist with weight loss.

### Description of the Related Art

Typical weight loss approaches fall into one of five categories: diet, exercise, pharmaceutical, surgical, and combinations thereof. Each of these categories has its own positives and negatives, along with the combinations providing new benefits and challenges.

Diet regimes, such as require keto or Atkins, requires substantial commitment to a restrictive diet, and to maintain the commitment for an extended period of time. The early stages of these diets can be particularly challenging for those undertaking them as the restrictive nature of the diet requires additional time commitments for determining which, and how much, foods should be consumed. Additionally, the “on diet” meals may be not to the palette of those undertaking the diet as the flavor profile is likely very different from the flavor profile of the typical meal consumed when not on the diet. While dieting, social events also present frequent, and occasionally awkward, challenges as it is frequent that dieting at a social event will be difficult at best. This results in convenient opportunities for “cheat days” which undermine the results of the diet.

Exercise requires substantial commitment in both financial and temporal terms. These are frequent barriers to those wanting to lose weight. Financial investments range from one-time costs such as home equipment to recurring costs such as gym memberships and things such as new athletic shoes. The temporal investment required to maintain the commitment required for weight loss from exercise alone is substantial and well-studied outside of this patent application. The personal discipline required to maintain the financial and temporal commitment to an exercise regimen, in the face of muscular soreness and overall fatigue, is another barrier to individuals wishing to achieve weight loss through exercise. Consequently, compliance with diet and exercise are traditionally the primary reasons that people who are overweight struggle to achieve and maintain healthier body weight.

Pharmaceutical use is likely the easiest approach to weight loss, as typical weight loss pharmaceuticals require doses taken by mouth daily. However, the most common weight loss pharmaceuticals, phentermine HCL and semaglutide, require prescription, physician monitoring, along with diet and exercise to show weight loss. Furthermore, pharmaceutical interventions are associated with potentially severe negative side effects.

Surgical methods for weight loss are numerous, with the most common being gastric bypass and gastric banding. These procedures involve invasive surgeries, which carry its own risk by itself, along with recovery time. Additionally, the surgeries are costly. Negative effects from the surgeries extend to dumping syndrome, in which food dumps from the stomach into the intestine before it has been property digested, osteoporosis, anemia, and even weight gain as a longer-term effect. Some reports show that as much as 85% of individuals after gastric bypass surgery suffer from some amount of dumping syndrome.

Stereotypically, men and women approach weight loss different. A 2017 study exploring gender differences in weight loss confirmed this. Stereotypically, men approach weight loss through primarily exercise and women approach weight loss through primarily dieting. However, despite similar weight loss needs, men are often underrepresented in weight control programs and trials, leading to less awareness of their benefits in men.

### SUMMARY

In accordance with the embodiments herein, a cannabinoid product to be consumed to assist with weight loss is described. The cannabinoid product described herein contains tetrahydrocannabivarin. Other cannabinoids may optionally be included as part of the product. The other cannabinoids are typically cannabidiol, cannabidivarin, cannabigerol, cannabidiolic acid, cannabigerolic acid, or cannabinol.

### DETAILED DESCRIPTION OF EMBODIMENTS

In the following description, for purposes of explanation and not limitation, details and descriptions are set forth in order to provide a thorough understanding of the invention. However, it will be apparent to those skilled in the art that the invention may be practiced in other embodiments that depart from these details and descriptions without departing from the spirit and scope of the invention.

The invention utilizes a variety of cannabinoids. The invention utilizes exclusively naturally occurring cannabinoids. Synthetic cannabinoids, such as those chemically synthesized from petroleum products, bacteria, enzymatic reactions, or other starting materials, are not considered and are not part of the invention, even if the same chemical compound. Extracted, naturally occurring cannabinoids are the exclusive area of the invention.

In an illustrative embodiment of the invention, the cannabinoid product is naturally occurring, and not synthetic, tetrahydrocannabivarin (“THCV”) consumed to assist with weight loss in the person consuming the THCV. In optional embodiments, the THCV may be combined with naturally occurring, and not synthetic, cannabidiol (“CBD”), and possibly even at least one assorted cannabinoids such as cannabidivarin (“CBDV”), cannabigerol (“CBG”), cannabidiolic acid (“CBDA”), cannabigerolic acid (“CBGA”), and/or cannabinol (“CBN”), where the assorted cannabinoids are naturally occurring, and not synthetic. Other cannabinoids are contemplated.

In a further embodiment of the invention, the cannabinoid product contains between 1.0 and 95.0 percent by mass of THCV and between 5.0 and 99.0 percent by mass of CBD. In the embodiments with the assorted cannabinoids, the THCV is typically between 1.0 and 70.0 percent by mass, the CBD is between 25.0 and 50.0 percent by mass, and the assorted cannabinoid each between 5.0 and 35.0 percent by mass.

Frequently, the invention is within a carrier oil. This carrier oil is typically a hemp oil, such as golden hemp seed oil. Occasionally, the invention would be an emulsion within the carrier oil, however this is not necessary. In an emulsion, water solubility and smaller particle sizes are more ideal for absorption of the invention into the human body. For the invention as an emulsion within the carrier oil, particle sizes between 50.0 nm and 5.0 μm are preferred but not necessary. Larger and smaller particle sizes are contemplated and still allow the invention to function.

Multiple types of delivery systems can be used with the invention. Some embodiments are as an oil, while others are tinctures, gel capsules, liquid emulsions, or even powders. Other types of delivery methods are contemplated.

Most typically, an individual will consume the invention to assist with weight loss. The use of the invention as a weight loss supplement has definitive advantages, namely that consumers are not expected to make major changes to their lifestyle or dietary habits when taking the invention. Unlike traditional appetite suppressant drugs, the invention appears to promote a homeostasis response in the body, resulting in modulated feedback to hunger and handling of caloric intake. Individuals taking the invention for at least 90 days reported steady weight loss, as well as reduced hunger and cravings, all without dietary restrictions or exercise regimens. By promoting natural, healthy weight loss, it is expected that participants will be able to integrate themselves into healthier habits without the traditional rebound effects seen in many diet or exercise programs. Such a program is likely to result in higher consumer compliance than any other weight loss program.

The clinical trial results related to the invention showed no significant difference in weight loss response between men and women. Men were less likely to use an organized weight loss program, and less likely to report it upon completion. It is important to recognize the benefits of weight loss, regardless of gender, for overall health improvement in overweight individuals. Because the incidence of obesity is nearly equal in men and women, weight loss should be handled in a way that is equally available and suitable to both men and women. The regulating effects of the invention showed no favoritism toward one gender, and as a result, the invention acts as an effective weight and health management tool for both men and women.

Individuals taking the invention for at least 90 days with no other direct change to lifestyle, including diet and exercise, achieved weight loss of between 10.0 and 30.0 pounds compared to the control group.

In addition to weight loss, while taking the invention individuals achieved reductions in body mass index as well as girth. While taking the invention for at least 90 days, individuals saw girth reduced by as much as 7.0 inches. BMI was reduced by as much as 8.0 in individuals taking the invention for at least 90 days. The BMI and girth reductions were seen with no other direct change in lifestyle, including diet and exercise.

In another illustrative embodiment, the invention is a cannabinoid product containing hemp seed oil and a mixture of cannabinoids made up of 74.0% CBD, 17.0% CBDV, 5.0% THCV, 2.0% CBG, 1.0% CBDA, 1.0% CBGA, and less than 0.1% CBN, with the percentages of each cannabinoid being percent by mass. This embodiment may be taken to assist with weight loss. It may be an emulsion; it may be an oil, tincture, gel capsule, or powder. Other forms of manufacture and delivery are contemplated.

What is claimed is:

1. A product, comprising:
tetrahydrocannabivarin; and
wherein the tetrahydrocannabivarin is used to assist with weight loss in an individual consuming the tetrahydrocannabivarin; and
wherein the tetrahydrocannabivarin is naturally occurring and not synthetic.

2. The product of claim 1, further comprising:
cannabidiol; and
wherein the cannabidiol is naturally occurring and not synthetic.

3. The product of claim 2, further comprising:
at least one assorted cannabinoid, wherein the at least one assorted cannabinoid is selected from the group consisting of cannabidivarin, cannabigerol, cannabidiolic acid, cannabigerolic acid, and cannabinol; and
wherein the at least one assorted cannabinoid is naturally occurring and not synthetic.

4. The product of claim 3, wherein
the tetrahydrocannabivarin is between 1.0 and 70.0 percent by mass;
the cannabidiol is between 25.0 and 50.0 percent by mass; and
the at least one assorted cannabinoid is between 5.0 and 35.0 percent by mass.

5. The product of claim 2, wherein;
the tetrahydrocannabivarin is between 1.0 and 95.0 percent by mass; and
the cannabidiol is between 5.0 and 99.0 percent by mass.

6. The cannabinoid product of claim 1, further comprising a carrier hemp oil.

7. The cannabinoid product of claim 1, wherein the cannabinoid product is prepared in a manner selected from the group consisting of oil, tincture, gel capsule, liquid emulsion, and powder.

8. The cannabinoid product of claim 1, wherein the cannabinoid product is an emulsion.

9. The cannabinoid product of claim 8, wherein the emulsion has a particle size between 50.0 nm and 5.0 um.

10. The cannabinoid product of claim 1, wherein the individual has achieved weight loss of at least 10.0 pounds after taking the cannabinoid product for at least 90 days.

11. The cannabinoid product of claim 1, wherein the individual has achieved weight loss of up to 30.0 pounds after taking the cannabinoid product for at least 90 days.

12. The cannabinoid product of claim 1, wherein the individual achieves weight loss without the aid of diet or exercise.

13. A cannabinoid product, comprising:
hemp seed oil; and
a cannabinoid mixture;
wherein the cannabinoid mixture comprises:
cannabidiol at 74.0 percent by mass;
cannabidivarin at 17.0 percent by mass;
tetrahydrocannabivarin at 5.0 percent by mass;
cannabigerol at 2.0 percent by mass;
cannabidiolic acid at 1.0 percent by mass;
cannabigerolic acid at 1.0 percent by mass; and
cannabinol at less than 0.1 percent by mass; and
wherein the cannabidiol, cannabivarin, tetrahydrocannabivarin, cannabigerol, cannabidiolic, cannabigerolic, and cannabinol are naturally occurring and not synthetic.

14. The cannabinoid product of claim 13, wherein an individual consumes the cannabinoid product to assist with weight loss.

15. The cannabinoid product of claim 14, wherein the individual has achieved weight loss of at least 10.0 pounds after taking the cannabinoid product for at least 90 days.

16. The cannabinoid product of claim 14, wherein the individual has achieved weight loss of up to 30.0 pounds after taking the cannabinoid product for at least 90 days.

17. The cannabinoid product of claim 13, wherein the individual achieves weight loss without the aid of diet or exercise.

18. The cannabinoid product of claim 13, wherein the cannabinoid product is prepared in a manner selected from the group consisting of oil, tincture, gel capsule, liquid emulsion, and powder.

19. The cannabinoid product of claim 13, wherein the cannabinoid product is an emulsion.

20. The cannabinoid product of claim 19, wherein the emulsion has a particle size between 50.0 nm and 5.0 um.
